(19) United States
(12) Patent Application Publication
(10) Pub. No.: US 2023/0076164 A1
(43) Pub. Date: Mar. 9, 2023

(54) Method for providing personalized cells with chimeric antigen receptors (CAR) against tumor microenvironment cells

(71) Applicant: Miltenyi Biotec B.V. & Co. KG, Bergisch Gladbach (DE)

(72) Inventors: Andreas Bosio, Bergisch Gladbach (DE); Olaf Thorsten Hardt, Bergisch Gladbach (DE); Mario Assenmacher, Bergisch Gladbach (DE)

(21) Appl. No.: 17/799,870

(22) PCT Filed: Jan. 15, 2021
(86) PCT No.: PCT/EP2021/050750

§ 371 Date: Aug. 15, 2022

(30) Foreign Application Priority Data
Feb. 17, 2020 (EP) 20157579.2

(51) Int. Cl. G01N 33/574; G01N 21/64

(52) U.S. Cl. CPC G01N 33/57492; G01N 21/6428; G01N 2021/6439

(57) ABSTRACT

The invention is directed to a process for providing a cell comprising a chimeric antigen receptor (CAR) specific for one or more target antigens exposed on tumor microenvironment cells characterized by providing a cell sample comprising tumor microenvironment cells and non-tumor microenvironment cells and repeating the steps of—contacting the cell tissue with a conjugate comprising a fluorescent moiety and an antigen recognizing moiety—removing unbound conjugate from the cell tissue and detecting cells bound to the conjugate by the fluorescence radiation emitted by the fluorescent moieties of the first conjugates—erasing the fluorescence emitted by the fluorescent moieties of the conjugates until identifying at least two conjugates provided with antigen recognizing moieties recognizing different antigens, allowing in combination to discriminate between tumor microenvironment cells and non-tumor microenvironment cells and providing cells with the identified at least two antigen recognizing moieties as chimeric antigen receptor (CAR). Preferable, the tumor microenvironment cells are tumor microenvironment cells from tumor stromal cells or PaCa cells.

### FIELD OF INVENTION

The present invention relates to a method for selecting antigens exposed on tumor microenvironment cells (TME), but not on healthy cells of an individual in order to provide cells with appropriate chimeric antigen receptors (CAR) for killing/lysing the tumor microenvironment cells (TME).

### BACKGROUND

Cancer is a broad group of diseases involving unregulated cell growth. In cancer, cells divide and grow uncontrollably, forming malignant tumors, and invading nearby parts of the body. The cancer may also spread to more distant parts of the body through the lymphatic system or bloodstream.

Solid tumors or cancers tissues are usually surrounded or encapsulated by tumor microenvironment cells (TME), which consists of blood vessels, immune cells, fibroblasts, pericytes, signaling molecules and the extracellular matrix (ECM). A definition of TME is disclosed in “NCI Dictionary of Cancer Terms”, National Cancer Institute, 2011-02-02. For a recent review on TME, see, Am J Cancer Res. 2019; 9(2): 228-241.

As CAR T cells are a promising immunotherapy to target tumors, treatment of solid tumors is affected by the TME, which can for example act as a physical barrier or be immunosuppressive. One potential way to address this is to again use CAR T cells.

The problem which arises is that the tumor and the TME are not the same for every patient and there is a need to select the right patient-specific targets for both the tumor and the TME and to generate CAR T cells (or the like) accordingly. Selection and generation of CART cells has to happen in a fast way and most of the time patient specific.

In praxis, one single target for a tumor cell or a cell of the TME might not be enough as a surface molecule can be expressed on non-target cells as well, thereby creating a safety problem. Furthermore, the surface molecule might not be selective enough, i.e. may be expressed on TME cells, leading surviving cells to relapse and/or leading to an incomplete TME destruction, and/or shut down the expression level of antigen and/or the affinity of a single binder might be too low to allow for efficient T cell activation.

In order to perform a personalized phenotyping of the tumor marker expression for an individual patient, combinations of CARs and CAR T cells need to be developed in an acceptable time frame which is dictated by the patient and the indication. In clinical praxis, an optimal time frame would be a couple of weeks.

Accordingly, the success of any immunotherapy for cancer based on CAR cells will rely on a fast and reliable selection of antigens specific for the respective tumor cells.

Such process is disclosed for example in EP3315511A1, wherein CAR T-cells are provided with antigen binding regions (“TCBM”) according to the immunological situation of the patient i.e. the antigens presented by the tumor cells.

It has been found that a distinct group of cell surface antigens is expressed on TME cells, but not or to a lower level on non-malignant cells. These antigens (also referred to as “markers”) can be used to identify and/or mark and/or destroy and/or disable escape mechanisms of TME cells via ligands that specifically bind to the markers. EP3315511A1 is silent on the identification of the most promising antigen binding regions (“TCBM”) for therapy. Further, EP3315511A1 is directed to tumor cells only and silent on the immunological situation of the TME.

After TME cells have been recognized by the CAR cells and the TME cells are thereby either destroyed or at least reduced in their efficiency to protect the cancer cells by the thus identifies CAR cells.

Object of the invention was therefore to provide a method to select antigens specific for TME cells, which are not expressed on healthy tissue in order to engineer cells which then kill/lyse cancer cells without attacking non-tumor cells.

### SUMMARY OF THE INVENTION

A first object of the invention is a process for providing a cell comprising a chimeric antigen receptor (CAR) specific for one or more target antigens exposed on tumor microenvironment cells characterized by providing a cell sample comprising tumor microenvironment cells and non-tumor microenvironment cells and repeating the steps of
contacting the cell tissue with a conjugate comprising a fluorescent moiety and an antigen recognizing moiety
removing unbound conjugate from the cell tissue and detecting cells bound to the conjugate by the fluorescence radiation emitted by the fluorescent moieties of the first conjugates
erasing the fluorescence emitted by the fluorescent moieties of the conjugates until identifying at least two conjugates provided with antigen recognizing moieties recognizing different antigens, allowing in combination to discriminate between tumor microenvironment cells and non-tumor microenvironment cells and providing cells with the identified at least two antigen recognizing moieties as chimeric antigen receptor (CAR).

Preferable, the method of the invention comprises repeating the above-mention steps with conjugates comprising a fluorescent moiety and antigen recognizing moieties recognizing different antigens at least for two subsequently performed cycle of steps.

In a first embodiment, the cell comprising a chimeric antigen receptor (CAR) is specific for one or more target antigens exposed on tumor microenvironment cells selected from the group consisting of alpha-SMA, CD74, CXCL12, and PDGFRbeta.

In a preferred variant of this embodiment, the cell comprising a chimeric antigen receptor (CAR) is specific for one or more target antigens exposed on tumor microenvironment cells and tumor stromal cells selected from the group consisting of alpha-SMA, CD74, CXCL12, and PDGFRbeta.

In a second embodiment, the cell comprising a chimeric antigen receptor (CAR) is specific for one or more target antigens exposed on tumor microenvironment cells selected from the group consisting of CD47, CD51, CD58, CD90, CD206 and CD239.

In a preferred variant of this embodiment, the cell comprising a chimeric antigen receptor (CAR) is specific for one or more target antigens exposed on tumor microenvironment cells and PaCa (pancreas carcinoma) cells selected from the group consisting of CD47, CD51, CD58, CD90, CD206 and CD239. The combination of CD90 and CD239 was found to be particular specific for tumor microenvironment cells of PaCa.

In a third embodiment, the cell comprising a chimeric antigen receptor (CAR) specific for one or more target antigens exposed on tumor microenvironment cells are further specific to tumor cells associated with the tumor microenvironment cells.

### BRIEF DESCRIPTION OF THE DRAWINGS

FIG. 1 shows the general domains of a chimeric antigen receptor (CAR)

FIG. 2 shows general building concepts of chimeric antigen receptors

FIG. 3 shows schematic the function of a CAR-T cell provided with an AND logic

FIG. 4 shows schematic the function of a CAR-T cell provided with an NOT logic

FIG. 5 a-c shows target identification by ultra-high-content imaging screening on pancreatic cancer

### DEFINITIONS

In the following, the terms “tumor microenvironment cells”, “TME cells” and “target cells” are used synonymously for the cells which shall be recognized (and later on attacked) by the CAR cells provided by the method of the invention.

Such tumor microenvironment cells (TME) grow around or embed a solid tumor and may consist of blood vessels, immune cells, fibroblasts, pericytes, signaling molecules and the extracellular matrix (ECM). A scientific definition may be found in “NCI Dictionary of Cancer Terms”. National Cancer Institute. 2011-02-02, for recent review see Am J Cancer Res. 2019; 9(2): 228-241).

Accordingly, the terms “non-tumor microenvironment cells”, “non-TME cells” and “non-target cells” are used synonymously for healthy cells which shall not be recognized (and later on attacked) by the CAR cells provided by the method of the invention.

The term “tumor cells” refers to cancer cells which are not part of the “tumor microenvironment cells” and of course are not healthy “non-tumor microenvironment cells”.

The term “Chimeric Antigen Receptor” or “CAR” refers to engineered cell which are provided with an new specificity originating from other sources than the cell itself.

The term “tumor” is known in medicine as “neoplasm”. Not all tumors are cancerous; benign tumors do not invade neighboring tissues and do not spread throughout the body.

The term “cancer” refers to a broad group of diseases involving unregulated cell growth. Cancerous cells divide and grow uncontrolled, forming malignant tumors, and invading nearby parts of the body. The cancer may also spread to more distant parts of the body through the lymphatic system or bloodstream.

The terms “binder” or “specifically binds” or “specific for” with respect to an antigen-binding domain of a ligand like an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refers to cells, preferentially T cells which are manipulated by recombinant methods well known in the art to express stably or transiently peptides or proteins which are not expressed in these cells in the natural state. For example, T cells are engineered to express an artificial construct such as a chimeric antigen receptor on their cell surface. For example, the sequences encoding the CAR may be delivered into cells using a retroviral or lentiviral vector.

The term “target” as used herein refers to an antigen or epitope associated with a cell that should be recognized specifically by an antigen binding domain, e.g. an antigen binding domain of an antibody or of a CAR. The antigen or epitope for antibody recognition can be bound to the cell surface but also be secreted, part of the extracellular membrane, or shed from the cell.

The term “antibody” as used herein refers to polyclonal or monoclonal antibodies and fragments thereof, which can be generated by methods well known to the person skilled in the art. The antibody may be of any species, e.g. mice, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “killer cell” as used herein refers to a cell that can kill/destroy/lyse another cell, e.g. a cancer cell. Most frequently, T cells, NK cells, dendritic cells and macrophages can be used as killer cells.

The term “engineered killer cell” as used herein refers to a killer cell that is genetically modified to allow for the specific killing of a target cell, e.g. a cell modified with a CAR against a target to kill tumor and/or TME cells expressing the respective target.

### DETAILED DESCRIPTION OF THE INVENTION

The process of the invention enables rapid identification of suitable sets of conjugates and subsequent rapid engineering of appropriate CAR cells in order to destroy at least a part of TME cells of a cell sample. The cell sample comprising TME cells, non-TME cells and non-tumor cells preferably origins from the same tissue or organ from the same patient.

In order to distinguish TME and non-TME cells in a cell sample, a skilled person like a pathologist may decide according to criteria known or lege antis in oncology whether the cell sample contains a tumor and if yes, what part of the cell sample. Such criteria are for example the guidelines published by the Arbeitsgemeinschaft der Wissenschaftlichen Medizinischen Fachgesellschaften (AWMF) and may involve e.g. the morphology of cells, structure of tissue, ploidity, biomarker expression (such as Her2, p53, BRAC1, APC, EGFR etc).

Since a tumor might be destroyed even if only a part of the TME cells are identified or attacked, it is not necessary that the conjugates identified bind to all TME cells. Preferable, the process of the invention is performed until identifying at least two conjugates provided with antigen recognizing moieties recognizing different antigens, binding to at least 1%, more preferred at least 10% and most preferred at least 30% of the TME cells.

While it is desired that the conjugates bind to 0% of the non-TME or non-tumor cells, it is acceptable for therapy that a certain amount of non-tumor/non-TME cells (i.e. healthy cells) are targeted. Preferable, the process of the invention is performed until identifying at least two conjugates, at least provided with antigen recognizing moieties recognizing different antigens, binding to TME cells and to at most 10%, more preferred to at most 5% and most preferred to at most 1% of the non-TME cells of the same type of tissue or organ. The term “same type of tissue or organ” refers for example to pancreas, liver or stroma.

The process of the invention enables the rapid identification of one or more sets of two conjugates provided with antigen recognizing moieties recognizing different antigens and subsequent one or more different cells with the identified at least two with antigen recognizing moieties as chimeric antigen receptor (CAR). Providing more than one set of conjugates and more than one CAR cell enhances chances of destroying TME cells and/or prevent TME cells downregulating antigens to escape being targeted.

The cell comprising a chimeric antigen receptor (CAR) may be selected from the group consisting of T-cells, NK-cells, or cells engineered to destroy (lyse) other cells when triggered by binding to the other cell.

In the process of the invention, the cell sample may be contacted subsequently with a plurality of binders to characterize the recognition pattern and to select binders specific for the TME-cells and binders specific for non-TME cells. The thus identified binders can be used for subsequent therapy as specified later.

In a variant of the process, at least two conjugates provided with antigen recognizing moieties recognizing different antigens bind to at least 20% of the TME cells and less than 5% of the non-TME cells of the cell sample. Preferable, at least two conjugates provided with antigen recognizing moieties recognizing different antigens bind to at least 50% of the TME cells and less than 5% of the non-TME cells of the cell sample. More preferable, at least two conjugates provided with antigen recognizing moieties recognizing different antigens bind to at least 70% of the TME cells and less than 5% of the non-TME cells of the cell sample.

In another variant, the at least two conjugates are provided with antigen recognizing moieties destroying at least 10 fold, preferable at least 50 fold, more preferable at least 100 fold more TME cells than non-TME cells of the cell sample.

The process of invention involves identifying at least two conjugates provided with antigen recognizing moieties recognizing different antigens, binding to TME cells but not or not essentially to non-TME cells.

If the CAR is provided with at least two antigen recognizing moieties, the CAR may be triggered to perform in different logic gates. The logic gates have been described in the literature as “AND”-, “NOT”-, “OR”-type CAR cells. As disclosed in more detail later, in the process of the invention, the at least two antigen recognizing moieties are provided as AND-, OR- or NOT-type and/or the at least two with antigen recognizing moieties are provided as ADAPTER-type. The at least two antigen recognizing moieties in all these variants are provided as chimeric antigen receptor (CAR).

### Chimeric Antigen Receptor (CAR)

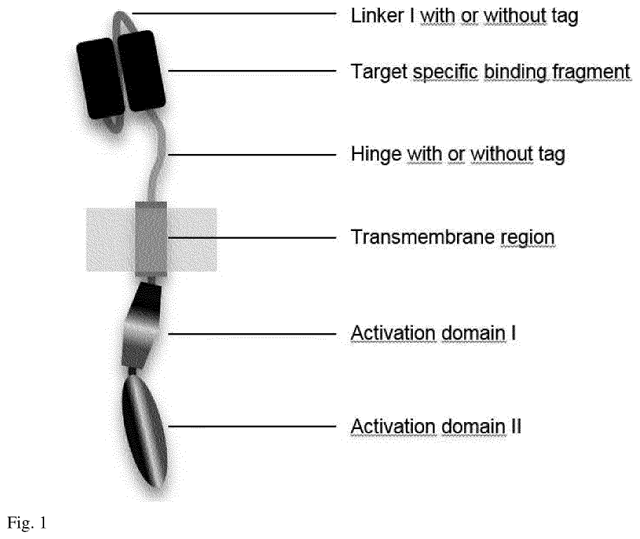

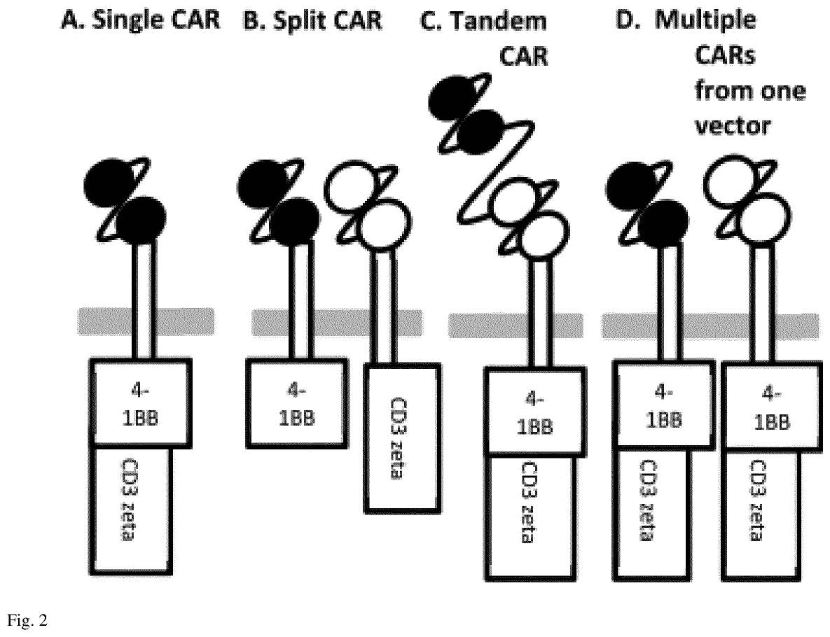

As shown in general in FIGS. 1 and 2, a chimeric antigen receptor (CAR) may comprise an extracellular domain comprising the antigen binding domain, a transmembrane domain and an intracellular signaling domain. The extracellular domain may be linked to the transmembrane domain by a linker. The extracellular domain may also be linked to a signal peptide.

The term “signal peptide” refers to a peptide sequence that directs the transport and localization of the protein within a cell, e.g. to a certain cell organelle (such as the endoplasmic reticulum) and/or the cell surface.

The term “antigen binding domain” refers to the region of the CAR that specifically binds to an antigen (and thereby is able to target a cell containing an antigen). The CARs obtained by the invention may comprise one or more antigen binding domains. Generally, the antigen binding domain on the CAR are extracellular. The antigen binding domain may comprise an antibody or a fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors can be used as an antigen binding domain. Often the antigen binding domain is a scFv. Normally, in a scFv the variable portions of an immunoglobulin heavy chain and light chain are fused by a flexible linker to form a scFv. Such a linker may be for example the “(G₄/S₁)₃-linker”.

The transmembrane domain of the CAR may for example comprise a sequence of the transmembrane domains of 4-1BB, CD8 and/or CD28. Further, an activating intracellular signaling domain may be present comprising a sequence of the intracellular signaling domains of CD28, CD137 or CD3zeta.

In alternative to an activating intracellular signaling domain, the CAR may comprise an inhibiting intracellular signaling domain having a sequence of the intracellular signaling domains such as PD1 or CTLA4.

In a preferred variant of this embodiment, the chimeric antigen receptor (CAR) comprises an antigen binding domain specific for CD20 without an additional antigen binding domain or additional CAR, wherein the antigen binding domain is conjugated to one transmembrane domains and one or more signaling domains. This variant is shown by way of example in FIG. 2A.

In some instances, it is beneficial for the antigen binding domain to be derived from the same species in which the CAR will be used in. For example, if the CAR is intended to be used therapeutically in humans, it may be beneficial for the antigen binding domain of the CAR to comprise a human or humanized antibody or fragment thereof. Human or humanized antibodies or fragments thereof can be made by a variety of methods well known in the art.

The terms “spacer” or “hinge” as used herein refers to the hydrophilic region which is between the antigen binding domain and the transmembrane domain. The CARs identified by the invention may comprise an extracellular spacer domain but is it also possible to omit such a spacer. The spacer may include Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR can be derived from any desired natural or synthetic source for such domain. If the source is natural the domain may be derived from any membrane-bound or transmembrane protein.

The cytoplasmic domain or the intracellular signaling domain of the CAR of the invention is responsible for activation of at least one of the normal effector functions of the immune cell in which the CAR is expressed. “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR of the invention to perform a specialized function. The intracellular signaling domain may include any complete or truncated part of the intracellular signaling domain of a given protein sufficient to transduce the effector function signal.

Prominent examples of intracellular signaling domains for use in the CARs include the cytoplasmic sequences of the T cell receptor (TCR) and co-receptors that act in concert to initiate signal transduction following antigen receptor engagement.

As for CARs with at least two antigen binding domains, the antigen binding domains may have different functions. The process of the invention reduces the time of development for new CAR cells suitable for tumor treatment which can be provided as “AND”-, “NOT”-, “OR”-type CAR cells.

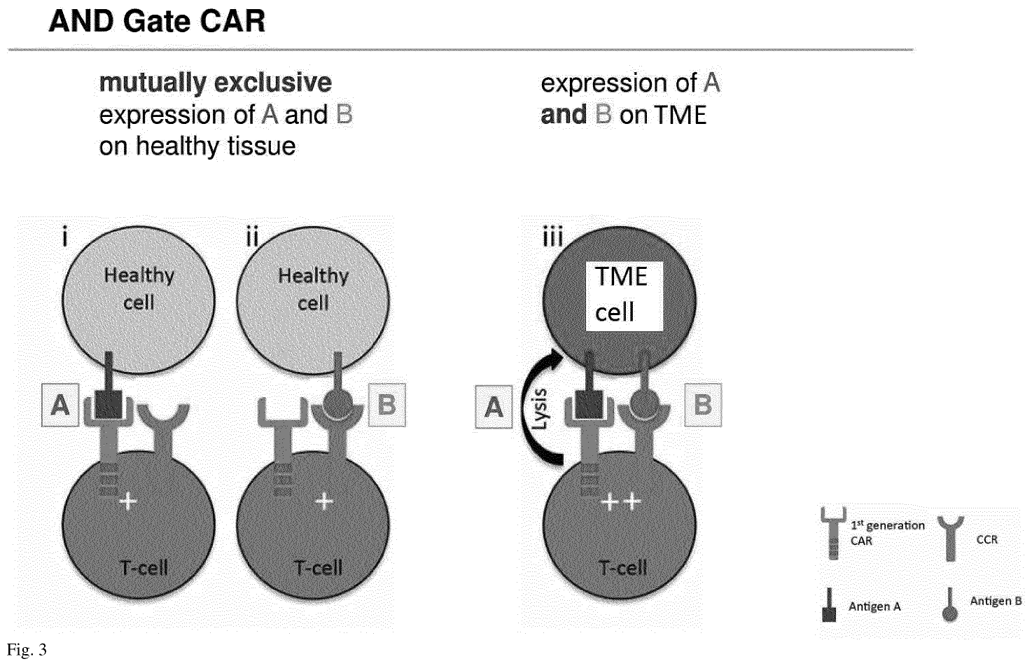

In one embodiment of the invention, the CAR cell will only be activated i.e. the signal of the signal domain will only be triggered when both antigen binding domains bind to their respective antigens. Such CAR cells are hereinafter referred to as “AND-CAR”. The antigen binding domains of “AND-CAR” cells may be identified by the method present invention by identifying for example two antigens located on TME cells of which non or at most one is expressed on healthy cells. FIG. 3 (published in Transl Cancer Res 2016;5(S1):S61-S65) shows how AND-CARs are deactivated by healthy cells (i) and (ii) but activated by TME cells (iii).

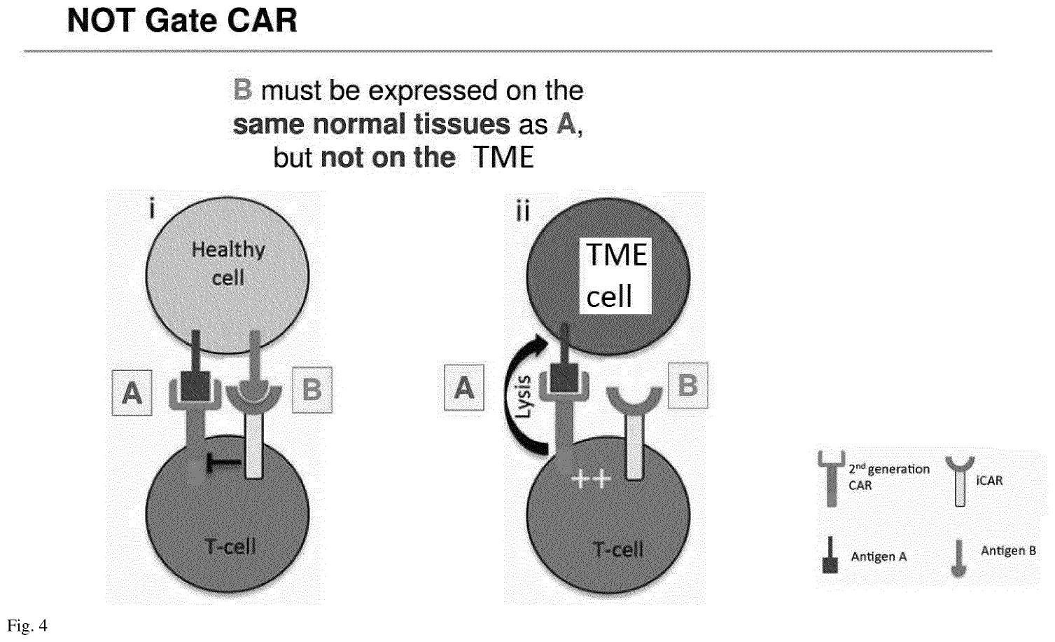

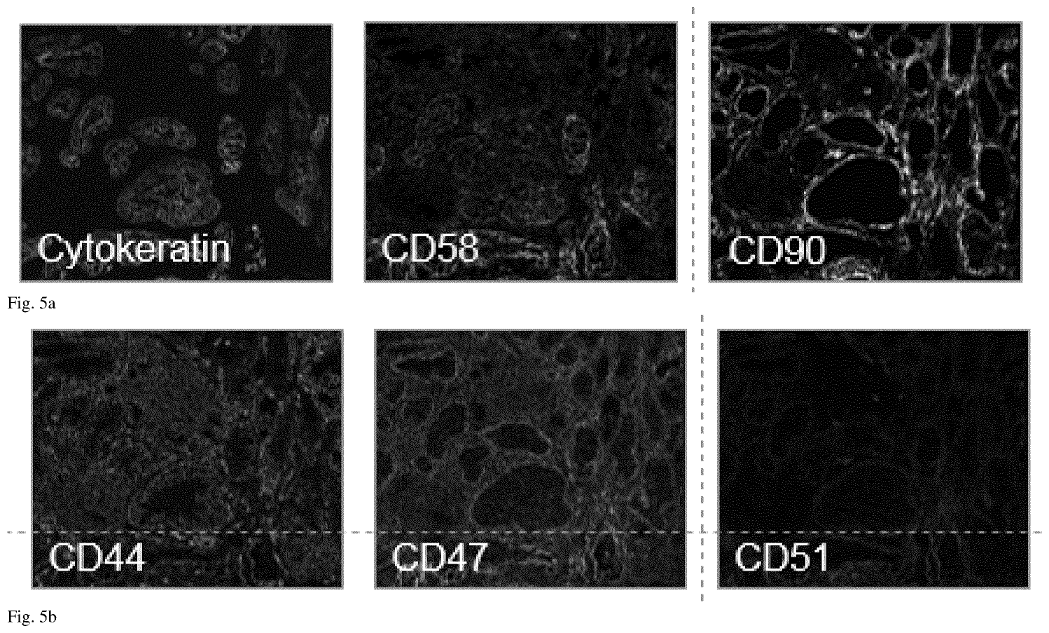

In another embodiment of the invention, the CAR cell will only be activated i.e. the signal of the signal domain will only be triggered when one antigen binding domains binds to its respective antigens and the other does not. Such CAR cells are hereinafter referred to as “NOT-CAR”. The antigen binding domains of “NOT-CAR” cells may be identified by the method present invention by identifying for example two antigens located on healthy cells, of which only one is expressed on TME cells. FIG. 4 (published in Transl Cancer Res 2016;5(S1):S61-S65) shows how NOT-CARs are deactivated by healthy cells (i) but activated by TME cells (ii.)

In yet another embodiment of the invention, the CAR cell will already be activated i.e. the signal of the signal domain will be triggered when only one of the two antigen binding domains binds to its respective antigens. Such CAR cells are hereinafter referred to as “OR-CAR”. The antigen binding domains of “OR-CAR” cells may be identified by the method present invention by identifying for example two antigens located on TME cells, but not on healthy cells.

A further reduction in processing time can be achieved by using so called “ADAPTER CAR” technology. An “ADAPTER CAR” cell comprises a signaling domain, a transmembrane domain and a linker domain which has no antigen recognizing properties. To this linker domain, one or more antigen recognizing moieties are then conjugated (linker plus molecule=adaptor). Accordingly, in the process according to the invention, the at least two with antigen recognizing moieties as chimeric antigen receptor (CAR) may be provided as ADAPTER-typ.

In a variant of the invention, the cells are provided with the identified at least two antigen recognizing moieties as chimeric antigen receptor (CAR) by providing a cell comprising biotin as antigen recognizing moiety of a chimeric antigen receptor (CAR) and providing conjugates of the identified at least two antigen recognizing moieties with an anti-Biotin moiety and conjugating said conjugates with said cells.

In another variant of the invention, the cell are provided with the identified at least two antigen recognizing moieties as chimeric antigen receptor (CAR) by providing a cell comprising biotin as antigen recognizing moiety of a first chimeric antigen receptor (CAR) and a second epitope as antigen recognizing moiety of a second chimeric antigen receptor (CAR) and providing conjugates of the identified at least two antigen recognizing moieties with an anti-Biotin moiety and a second moiety conjugating said conjugates with said cells.

Suitable linkers are molecules which are either not present in a mammalian (human) body or have a low affinity to naturally occurring antibodies in a mammalian (human) body. Suitable are for example biotin or thimanine units (chemically modified or unmodifies) biotin which are identified by an appropriate adapter like an anti-biotin or anti-thiamine antibody which is conjugated to the desired conjugated to the antigen recognizing moiety.

“ADAPTER CAR” technology is disclosed in WO2018078066 and uses the terms “linker/label epitope” (LLE) for the linker bound to the CAR cell

Preferable, the LLE does not evoke an immune reaction in a subject intended to be treated with cells expressing the CAR. This can be achieved by using an extracellular LLE binding domain of a CAR that is derived from an naturally occurring epitope recognizing molecule such that the LLE is bound with a higher preference than to the endogenous label moiety without linker moiety, i.e. the naturally occurring molecule in the subject (the self antigen).

For example, an extracellular LLE binding domain of a CAR binds with an at least twofold, preferentially at least 5-fold, more preferentially at least 10-fold higher affinity to said LLE than to the said naturally occurring molecule.

In the case of “ADAPTER” CAR cells, the CAR cell provided with the linker unit and the adaptor are applied in parallel to the patient. Advantage of this embodiment is that the CAR cell with a linker may be prepared independent of the identified antigen recognizing moieties and does not have to be developed from the scratch. Only the adaptor has to be generated and tested. This approach allows also to control the strength and kinetics of the therapy, similar to a conventional chemotherapy.

### Detecting Process

In the method of the invention, the fluorescence emitted by the fluorescent moieties of the conjugates bound to cells need to be erased. This step allows to investigate a plurality of potential binders or conjugates until the best combination of conjugates (i.e. antigen binding moieties i.e. antigens of the TME cells) is determined. The term “erasing the fluorescence” refers to any method which quenches fluorescence radiation of the cell sample and includes removal of the fluorescent moiety from the bound conjugate for example by enzymatically degrading the conjugates. Such conjugates are for example disclosed in EP3037821A1, EP16203689.1 or EP 16203607.3.

In a variant thereof, conjugates comprising an affinity system for example biotin/anti-biotin can be used, where the affinity system is abrogated by addition of a free competitor molecule (like biotin for an biotin/anti-biotin system). In any case, the fluorescence is erased by release of the fluorescence moiety from the conjugate and subsequent washing away the “released” fluorescence moiety.

In an alternative method for “erasing the fluorescence”, the fluorescence moiety is chemically destroyed to the extend of removing its capability of emitting fluorescence radiation by adding oxidative agents like hydrogen peroxide or providing radiation of an appropriate wavelength (like UV radiation).

### Use for Treatment of Cancer

The cell population obtained by the method of the invention may be used as pharmaceutical composition, preferable in a method for treatment of human cancer, especially human pancreas cancer or human stromal cancer.

The pharmaceutical composition comprises preferable a population of engineered cells expressing a CAR obtained by the method of the invention. In a variant of the invention, the pharmaceutical composition is used in combination with a chemotherapeutic, radiation, or immunomodulatory agent for treatment of cancer.

The recognition of the TME by the pharmaceutical composition may lead to a reaction such as the secretion of molecules by the CAR cells, which recruits further cells reducing the efficiency of the TME to protect the cancer cells. In the end, the cancer cells may be attacked for example by appropriate CAR cells.

The TME cells of the cancer to be treated may include hematopoietic cancer, myelodysplastic syndrome, pancreatic cancer, head and neck cancer, cutaneous tumors, minimal residual disease (MRD) in acute lymphoblastic leukemia (ALL), acute myeloid leukemia (AML), lung cancer, breast cancer, ovarian cancer, prostate cancer, colon cancer, melanoma or other hematological cancer and solid tumors, or any combination thereof. In another embodiment, the cancer includes a hematological cancer such as leukemia (e.g., chronic lymphocytic leukemia (CLL), acute lymphocytic leukemia (ALL), acute myeloid leukemia (AML), or chronic myelogenous leukemia (CML), lymphoma (e.g., mantle cell lymphoma, non-Hodgkin's lymphoma or Hodgkin's lymphoma) or multiple myeloma, or any combination thereof. Furthermore, the cancer may include an adult carcinoma comprising coral and pharynx cancer (tongue, mouth, pharynx, head and neck), digestive system cancers (esophagus, stomach, small intestine, colon, rectum, anus, liver, intrahepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, neuroblastoma, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

The treatment of cancer may encompass any method which involves at least one antigen as disclosed or any combination of antigens as disclosed as target molecule. Such methods may be e.g. treatment with agents which bind to the antigen and affect the viability of the cancerous cell expressing this antigen, preferentially kill the cancerous cell. Examples are oncolytic viruses, BiTEs®, ADCCs and immunotoxins as already disclosed.

For the treatment, immune cells, e.g. T cells of a subject may be isolated. The subject may suffer from said cancer or may be a healthy subject. These cells are genetically modified in vitro or in vivo to express one or more CARs of the invention. These engineered cells may be activated and expanded in vitro or in vivo. In a cellular therapy these engineered cells may be infused to a recipient in need thereof. These cells may be a pharmaceutical composition (said cell plus pharmaceutical acceptable carrier). The infused cells are able to kill (or at least stop growth of) cancerous cells expressing one or more of the disclosed antigens in the recipient. The recipient may be the same subject from which the cells was obtained (autologous cell therapy) or may be from another subject of the same species (allogeneic cell therapy).

### Examples

The following examples are intended for a more detailed explanation of the invention but without restricting the invention to these examples.

Fresh-frozen pancreatic carcinoma specimens were sliced and fixed with acetone. The subsequent screening was performed on the MACSimaTM ultra-high-content imaging platform (Miltenyi Biotec B. V. & Co. KG) by employing a sequential staining of 90 antibodies of which 9 are depicted; nuclei were stained with DAPI.

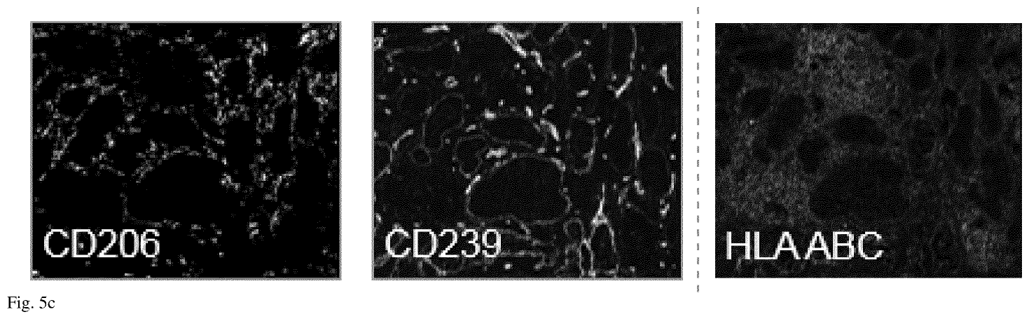

In FIG. 5, it is shown that combinations of the markers CD47, CD51, CD58, CD90, CD206 and CD239, could be used to distinguish TME and tumor cells against healthy cells. In particular, the combination of CD90 and CD239 was specific for the tumor microenvironment cells as both show overlapping expression on cells of stromal identity but not on other cell types. When combining these markers with markers or marker combinations specific for tumor cells, one could specifically attack TME and optionally the tumor.

1. Process for providing a cell comprising a chimeric antigen receptor (CAR) specific for one or more target antigens exposed on tumor microenvironment cells characterized by providing a cell sample comprising tumor microenvironment cells and non-tumor microenvironment cells and repeating the steps of
contacting the cell tissue with a conjugate comprising a fluorescent moiety and an antigen recognizing moiety
removing unbound conjugate from the cell tissue and detecting cells bound to the conjugate by the fluorescence radiation emitted by the fluorescent moieties of the first conjugates
erasing the fluorescence emitted by the fluorescent moieties of the conjugates until identifying at least two conjugates provided with antigen recognizing moieties recognizing different antigens, allowing in combination to discriminate between tumor microenvironment cells and non-tumor microenvironment cells and providing cells with the identified at least two antigen recognizing moieties as chimeric antigen receptor (CAR).

2. Process according to claim 1, characterized in that the cell comprising a chimeric antigen receptor (CAR) is selected from the group consisting of T-cells, NK-cells, or cells engineered to destroy other cells when triggered by binding of the other cell.

3. Process according to claim 1, characterized in that the at least two antigen recognizing moieties are provided as AND-, OR- or NOT-type.

4. Process according to claim 1, characterized in that the at least two antigen recognizing moieties are provided as ADAPTER-type.

5. Process according to claim 4, characterized in that the cell are provided with the identified at least two antigen recognizing moieties as chimeric antigen receptor (CAR) by providing a cell comprising biotin as antigen recognizing moiety of a chimeric antigen receptor (CAR) and providing conjugates of the identified at least two antigen recognizing moieties with an anti-Biotin moiety and conjugating said conjugates with said cells.

6. Process according to claim 4, characterized in that the cell are provided with the identified at least two antigen recognizing moieties as chimeric antigen receptor (CAR) by providing a cell comprising biotin as antigen recognizing moiety of a first chimeric antigen receptor (CAR) and a second epitope as antigen recognizing moiety of a second chimeric antigen receptor (CAR) and providing conjugates of the identified at least two antigen recognizing moieties with an anti-Biotin moiety and a second moiety conjugating said conjugates with said cells.

7. Process according to claim 1, characterized in that the at least two conjugates provided with antigen recognizing moieties binding at least at least 20% of the tumor microenvironment cells and less than 5% of the non-tumor microenvironment cells.

8. Process according to claim 1, characterized in that the at least two conjugates provided with antigen recognizing moieties binding at least 10 fold more tumor microenvironment cells than non-tumor microenvironment cells of the cell sample.

9. Process according to claim 1, characterized in the fluorescence radiation emitted by the fluorescent moieties is erased by enzymatically degrading of the conjugates.

10. Process according to claim 1, characterized in the cell comprising a chimeric antigen receptor (CAR) specific for one or more target antigens exposed on tumor microenvironment cells are further specific to tumor cells associated with the tumor microenvironment cells.

11. Process according to claim 1, characterized in that the cell comprising a chimeric antigen receptor (CAR) is specific for one or more target antigens exposed on tumor microenvironment cells selected from the group consisting of alpha-SMA, CD74, CXCL12, and PDGFRbeta.

12. Process according to claim 11, characterized in that the cell comprising a chimeric antigen receptor (CAR) is specific for one or more target antigens exposed on tumor microenvironment cells and tumor stromal cells selected from the group consisting of alpha-SMA, CD74, CXCL12, and PDGFRbeta.

13. Process according to claim 1, characterized in that the cell comprising a chimeric antigen receptor (CAR) is specific for one or more target antigens exposed on tumor microenvironment cells selected from the group consisting of CD47, CD51, CD58, CD90, CD206 and CD239.

14. Process according to claim 13, characterized in that the cell comprising a chimeric antigen receptor (CAR) is specific for one or more target antigens exposed on tumor microenvironment cells and PaCa cells selected from the group consisting of CD47, CD51, CD58, CD90, CD206 and CD239.

15. Process according to claim 10, characterized by providing a cell sample comprising tumor microenvironment cells, tumor cells and non-tumor microenvironment cells and repeating the steps of
contacting the cell tissue with a conjugate comprising a fluorescent moiety and an antigen recognizing moiety
removing unbound conjugate from the cell tissue and detecting cells bound to the conjugate by the fluorescence radiation emitted by the fluorescent moieties of the first conjugates
erasing the fluorescence emitted by the fluorescent moieties of the conjugates until identifying at least two conjugates provided with antigen recognizing moieties recognizing different antigens, allowing in combination to discriminate between tumor microenvironment cells and tumor cells against non-tumor microenvironment cells and providing cells with the identified at least two antigen recognizing moieties as chimeric antigen receptor (CAR).
